(19) United States
(12) Patent Application Publication
(10) Pub. No.: US 2012/0217189 A1
(43) Pub. Date: Aug. 30, 2012

(54) DEVICE FOR EXTRACORPOREAL BLOOD TREATMENT

(76) Inventors: Jörn Ahrens, Baunatal (DE); Stefan Moll, Melsungen (DE); Alex Castellarnau, Melsungen (DE)

(21) Appl. No.: 13/394,021

(22) PCT Filed: Sep. 6, 2010
(86) PCT No.: PCT/EP10/05455

§ 371 Date: May 15, 2012

(30) Foreign Application Priority Data
Sep. 4, 2009 (DE) 102009040104.0

(51) Int. Cl. A61M 1/16

(52) U.S. Cl. 210/96.2

(57) ABSTRACT

The invention is related to an apparatus for extracorporeal treatment of blood with a dialyzer which is separated by a semipermeable membrane into a first and a second chamber, wherein the first chamber is disposed in a dialysis fluid pathway and the second chamber is connectable to the blood circulation of a patient by means of a blood inflow conduit and a blood outflow conduit, a feed for fresh dialysis fluid, a discharge for spent dialysis fluid, a means for estimation of a flow Q_{Blood} in the blood inflow conduit respectively the blood outflow conduit, a means for determining a flow Q_{Dialysate} in the dialysis fluid pathway, and a measuring device disposed in the discharge for estimation of the absorption A_{Dialysate} of the spent dialysis fluid flowing through the discharge, wherein the measuring device has at least one radiation source and a detector system for detection of the intensity of the electromagnetic radiation. Therein provided are a memory, wherein a formula

$${A}_{Blood} = ( \frac{{A}_{Dialysate} \cdot {Q}_{Dialysate}}{{Q}_{Blood}} )$$

is stored for the absorption A_{Blood}, and an arithmetic unit for estimation of the absorption A_{Blood} in the blood inflow conduit respectively the blood outflow conduit using the absorption A_{Dialysate}, the flow Q_{Dialysate}, and the flow Q_{Blood}.

The invention is related to an apparatus for the extracorporeal treatment of blood according to the generic term of claim 1.

In patients with reduced or no renal function at all, waste products including toxic substances are removed by a kidney replacement therapy, wherein the blood of the patient is fed from the patient to the artificial kidney, respectively the dialyzer, by a blood supply conduit. Inside the artificial kidney, respectively the dialyzer, the blood of the patient is brought into contact with the dialysis fluid via a semi-permeable membrane. The dialysis fluid contains different salts in such a concentration that the waste products including the toxic substances are transferred from the blood of the patient into the dialysis fluid by diffusion and convection. The blood such cleaned from the waste products is fed back into the blood circulation of the patient via a blood outflow conduit connected to the dialyzer.

For quantification of the result of the kidney replacement therapy it is necessary to control the efficiency of the kidney replacement therapy directly respectively online. Therefore, the so-called Kt/V model was developed. Therein the Kt/V-value is a parameter for estimation of the efficiency of a kidney replacement therapy, wherein the clearance K stands for the volume flow of the purified uremic substances, t for the time of treatment, and V for the volume of distribution of the patient. Thereby, K as well as V is related each to the particular waste product. Usually, the efficiency of a kidney replacement therapy is described using urea as a waste product, so that K describes the urea clearance and V the distribution volume of urea in the patient, which basically corresponds to the body water of the patient.

From EP1083948A1 it is known to estimate the Kt/V value respectively the reduction rate RR for a particular waste product during the kidney replacement therapy spectrophotometrically by means of a measuring device located in the discharge using UV radiation and its absorption by substances obligatory excreted by urine in the dialysis fluid. For estimation of this Kt/V value respectively the reduction rate it is necessary to know the concentration respectively the proportional absorption of substances obligatory excreted by urine in the blood of the patient to be treated at any point of time during the kidney replacement therapy. For estimation of this absorption it is suggested in EP2005982A1 to recirculate the dialysis fluid during the kidney replacement therapy against the extracorporeal blood stream of the patient as long until an equilibration of the concentrations and thus a constant value of the absorption of substances obligatory excreted by urine in the dialysis fluid has been reached. Once this constant value has been reached the concentration of the substances obligatory excreted by urine in the dialysis fluid corresponds to the concentration of the substances obligatory excreted by urine in the blood of the patient to be treated, so that the estimated absorption in the dialysis fluid be equaled to the one in the blood of the patient. By this measure instead of the Kt/V-value a so called equilibrated Kt/V-value eKt/V is estimated, so that the equilibration of the concentration of the substances obligatory excreted by urine within the different compartments of the patient body is considered.

However, it has been shown to be problematic in this method, that by carrying out the recirculation of the dialysis fluid against the extracorporeal blood stream of the patient the duration of therapy is considerably prolonged due to the declining concentration gradient during the operation, which follows the run of a charging function, because reaching the concentration equilibration can take five minutes of also longer. If this procedure is carried out more than once during the treatment of a patient the duration of therapy prolongs accordingly, which is not only uneconomical but also means additional stress for the patient. Furthermore it is additionally problematic that in this method the full amount of substances obligatory excreted by urine present in the blood and thus a correspondingly high absorption has to be estimated, which exceeds the measuring range appearing in therapy by the factor 3, since there is no dilution. Insofar additional extensive technical equipment is also necessary regarding the used device for measuring the absorption for estimation of the occurring absorptions during the therapy itself and during the recirculation of the dialysis fluid.

It is the object of the present invention to provide an apparatus which makes it possible to estimate the appropriate values of concentration respectively absorption without extending the duration of therapy respectively treatment of the patient, so that the therapy can be carried out not only more economically, but an additional stress of the patient by longer taking therapies respectively treatments would also be minimized. Therein the equipment requirements with regard to the device measuring the absorption can be kept at a minimum.

This object is solved by an apparatus with the features of claim 1. Advantageous forms of the inventions are subject matter of the dependent claims.

Initiation point of the present invention is the following context between the dialyzer clearance Q_{clearance} and the blood flow Q_{Blood} during a kidney replacement therapy as found by Kaufmann et al. (“Solute disequilibrium and multicompartment modeling”, Advances in renal replacement therapy 1995; 2(4):319-29, Kaufmann A M; Schneditz D; Smye S; Polaschegg H D; Levin N W):

$${Q}_{clearance} = {Q}_{Blood} ⊙ ( 1 - {e}^{[ - \frac{P \cdot S}{{Q}_{Blood}} ]} ) \qquad ( 1 )$$

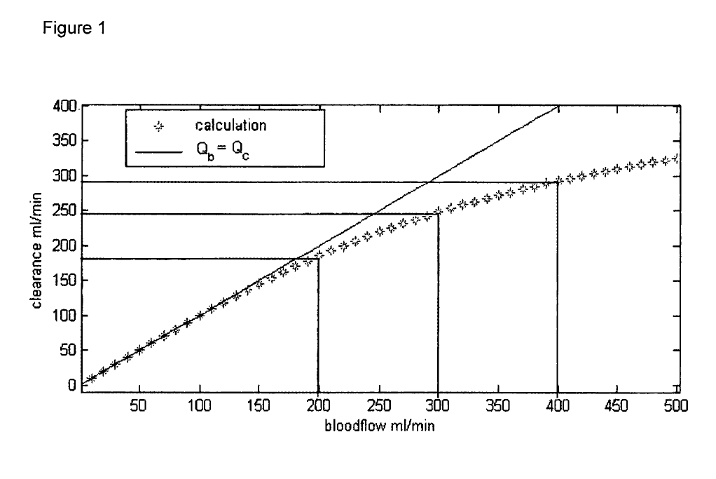

Therein P is referring to the permeability and S to the surface of the membrane material of a dialyzer used for a kidney replacement therapy. Therein the product P⊙S is a feature of the material which is characteristic for each dialyzer usually shown in the data sheets of the manufacturer respectively can be estimated on the basis of the data presented therein. In the present example as shown in FIG. 1 the estimation was done with a P⊙S value of 525 ml/min which corresponds to a commercially used dialyzer.

As can be seen in formula (1), a high value for P⊙S and a low value for the blood flow Q_{Blood} will lead to a linear function between clearance and blood flow at low blood flows, because the part of the e-function is approaching zero. Formula (1) shows therefore that at blood flows <100 ml/min the deviation between blood flow and clearance is so low that it can be neglected and thus the blood flow Q_{blood} equals the clearance Q_{clearance}.

With the inventive apparatus it is now possible to estimate an absorption A_{Blood} using the amounts of toxic substances during a kidney replacement therapy, as laid out in the following:

During the measurement the absorption A_{Dialysate} is measured on the side of the dialysis fluid beyond the dialyzer. Since the transport of the amounts of blood through the dialyzer has to be equal to that of the dialysis fluid, this results in following mathematical context as described in formula (2a) and (2b)

$$\begin{matrix}{A}_{Blood} ⊙ {Q}_{clearance} = {A}_{Dialysate} ⊙ {Q}_{Dialysate} & ( 2 a ) \\ {A}_{Blood} = ( \frac{{A}_{Dialysate} \cdot {Q}_{Dialysate}}{{Q}_{clearance}} ) & ( 2 b )\end{matrix}$$

If the value of the dialyzer clearance of formula (1) is set into formula (2b), one obtains formula (2c):

$${A}_{Blood} = ( \frac{{A}_{Dialysate} \cdot {Q}_{Dialysate}}{{Q}_{Blood} ⊙ ( 1 - {e}^{[ - \frac{P \cdot S}{{Q}_{Blood}} ]} )} ) \qquad ( 2 c )$$

With known values for the product P⊙S the absorption A_{Blood} can be estimated at any point of time of the therapy by measuring the absorption A_{Dialysate} in the discharge of dialysis fluid, the dialysate flow Q_{Dialysate} and the blood flow Q_{Blood}. By using this Q_{Blood}-value it is now possible to estimate the equilibrated Kt/V-value at any point of time of the therapy without substantial delay, so that the duration of therapy is minimized using the inventive apparatus.

In order not to depend always on the product P⊙S of the used dialyzer it has been shown to be advantageous to set the value for the e-function in the formulas (1) and (2c) to zero. This makes particular sense because that way the estimation of the absorption A_{Blood} is independent from the used dialyzers. The deviation at a blood flow of 100 ml/min and a P⊙S-value of 500 is thereby already less that 1%, which can be worked with out any problems.

That way formula (1) is resulting in:

Q_{Clearance}=Q_{Blood} (1b)

and formula (2c) in:

$${A}_{Blood} = ( \frac{{A}_{Dialysate} \cdot {Q}_{Dialysate}}{{Q}_{Blood}} ) \qquad ( 2 d )$$

And with formulas (2c or 2d) the absolute absorption on the blood side can be estimated. Thereby the rate of ultrafiltration has to be reduced to a minimum in order to minimize its influence on the measurement of absorption.

For comparison of the single measurement with other measurements, particularly during other therapeutic treatments, respectively for making possible a comparison at variable dialysis fluid flows or ultrafiltration flows during the same therapeutic treatment, it has been shown that it is helpful and advantageous to standardize the estimated absorption in the dialysate to a standard flow of e.g. 500 ml/min. The formal context for the standardized absorption A_{standard} is resulting in:

$${A}_{Standard} = {A}_{Dialysate} ⊙ ( \frac{{Q}_{Dialysis fluid} + {Q}_{UF}}{{Q}_{Standard}} ) \vert {Q}_{Standard} = 500 ml / min \qquad ( 3 )$$

By using the estimation of A_{Blood} using the formulas 2c or 2d of A_{Blood} it is possible to estimate the absorption respectively the corresponding proportional concentration of the substances obligatory excreted by urine in the blood side without making measurements directly in the blood stream or by taking blood samples.

Thus at low blood flows it is possible to make the assumption Q_{Blood}=Q_{Clearance}, which makes it possible to estimate the absorption on the blood side using formula (2c). In parallel formula (1) can of course be used directly in formula (2d) when knowing “P⊙S”.

Then with the help of absorption A_{Blood} on the blood side the estimation of the eKT/V can be carried out by using the water volume of the patient and the integration of the absorption via the dialysate flow. As far as the medium molecular proportion in the blood has no influence on the spectrum of the absorption measurement, the eKt/V can be estimated using the following formula (4e), as well. As far as changes of the flow occur, for example by the UF-rates, this can be compensated by the standardization.

$$\begin{matrix}Amount of {substance}_{Blood\_Starting point} = ( {A}_{Blood\_Starting point} ⊙ {V}_{Starting point} ) & ( 4 a ) \\ Amount of {substance}_{Dialysate} = \int ( {A}_{Dialysate} ( t ) ⊙ {Q}_{Dialysate} ( t ) ) d t & ( 4 b ) \\ {A}_{Blood\_End} of therapy = ( \frac{\begin{matrix}Amount of {substance}_{Blood\_Starting point} - \\ Amount of {substance}_{Dialysate}\end{matrix}}{{V}_{Starting point} - {V}_{UF}} ) & ( 4 c ) \\ Amount of {substance}_{Blood\_End of therapy} = Amount of {substance}_{Blood\_Starting point} - Amount of {substance}_{Dialysate} & ( 4 d ) \\ eKt / V = - ln ( \frac{{A}_{Blood\_End of therapy}}{{A}_{Blood\_Starting point}} ) & ( 4 e )\end{matrix}$$

In parallel it is possible to estimate the absolution proportion of the medium molecular substances in the blood by way of adding the convective part during the measurement of absorption using the UF-pump. For this purpose first a measurement is carried out with a pure diffusive proportion, and then the absorption of the small molecular substances is estimated with the help of a standardized absorption of the dialysis fluid (5a) by setting it into the formula (5d).

Subsequently a new measurement is carried out by connecting the UF-pump, and a standardization (5b) of the absorption to the flow is carried out, as well, because the flow is increasing by the addition of the ultrafiltration flow.

The medium molecular proportion can be estimated by formula (5c) using both measurements. The basis for doing this is that the measurement of the diffusive proportion has been carried out with a lower blood flow, so that the amount of small molecular substances is transferred from the blood into the dialysis fluid completely (Q_{Blood}=Q_{Clearance}), because otherwise yet present diffusively transported substances could cause a falsification upon the addition of the convective transport, if this could not be compensated by a mathematical correction.

The estimation of the absorption of the medium molecular substances on the blood side is done by formula (6a). Because only a part of the medium molecules of the blood flow Q_{blood} is removed by the UF-rate in contrast to the diffusive transport, the absorption value (6a) is standardized for the complete blood flow.

$$\begin{matrix}{A}_{Standardized} = ( {A}_{Dialysate} ⊙ \frac{{Q}_{Dialysate}}{{Q}_{Standard}} ) \vert {Q}_{Standard} = 500 ml / min & ( 5 a ) \\ {A}_{Standardized + UF} = ( {A}_{Dialysate + UF} ⊙ \frac{{Q}_{Dialysate} + {Q}_{UF}}{{Q}_{Standard}} ) \vert {Q}_{Standard} = 500 ml / min & ( 5 b ) \\ {A}_{Dialysate\_MM} = ( {A}_{standardized + UF} - {A}_{standardized} ) ⊙ & ( 5 c ) \\ {A}_{Blood} = ( \frac{{A}_{Standardized} \cdot {Q}_{Dialysate}}{{Q}_{Blood}} ) \vert {Q}_{Clearance} = {Q}_{Blood} & ( 5 d ) \\ {A}_{Blood\_MM} = ( {A}_{standardized + UF} - {A}_{standardized} ) ⊙ ( \frac{{Q}_{Blood}}{{Q}_{UF}} ) & ( 6 a )\end{matrix}$$

In parallel the eKt/V (6f) can also be estimated for the medium molecular proportion by estimation of the medium molecular proportion (6c) removed by activation and deactivation of the UF-pump and then calculation it with the absolute medium molecular proportion (6b) in the blood of the patient estimated at the beginning (formulas 6a-6f):

$$\begin{matrix}Amount of {substance}_{Blood\_MM \_Starting point} = ( {A}_{Blood\_MM \_Starting point} ⊙ {V}_{Starting point} ) & ( 6 b ) \\ Amount of {substance}_{Dialysate\_MM} = \int ( {A}_{Dialysate\_MM} ( t ) ⊙ {Q}_{Standard} ) d t & ( 6 c ) \\ {A}_{Blood\_MM \_End of therapy} = ( \frac{\begin{matrix}Amount of {substance}_{Blood\_MM \_Starting point} - \\ Amount of {substance}_{Dialysate\_MM}\end{matrix}}{{V}_{Starting point} - {V}_{UF}} ) & ( 6 d ) \\ Amount of {substance}_{Blood\_MM \_End of theapy} = Amount of {substance}_{Blood\_MM \_Starting point} - Amount of {substance}_{Dialysate\_MM} & ( 6 e ) \\ eKt / {V}_{convection} = - ln ( \frac{{A}_{Blood\_MM \_End of therapy}}{{A}_{Blood\_MM \_Starting point}} ) & ( 6 f )\end{matrix}$$

For estimation of the diffusive proportion over the integral over the therapy thus a phase of measuring with a pure diffusive proportion without ultrafiltration has to be introduced during the therapy.

For estimation of the pure diffusive proportion the ultrafiltration rate is reduced to the minimum, the blood flow is adapted and the value of absorption at this point of time is registered. For estimation of the convective proportion subsequently the convective proportion is added again in order to estimate the absorption of the convective proportion out of the difference (5c).

Thus it is possible to estimate the Kt/V respectively the eKt/V on the basis of the fitted progress of the diffusive absorption (5a) over the therapy, the convective progress of the absorption (5c) and the progress of the absorbance itself out of convective and diffusive proportions. As far as the set ultrafiltration corresponds during the normal measuring phase to the normal UF-rate of the therapy it can be maintained as long as the difference of the signals is sufficient. The relation of the diffusive phase (UF-rate=0) and the convective phase (UF-rate on) should ideally correspond to the standardized UF-rates set in the therapy over the period of measurement in order to prevent further adaptations for reaching the target volume.

For estimation of an effective clearance the absorbance A_{Blood} has to be estimated without ultrafiltration first. Then it is possible to estimate the clearance again in the normal therapy also directly thereafter without ultrafiltration. Under the assumption that thereafter the value A_{Blood} is still present in the blood in the same concentration, the effective clearance can be estimated using the formula (7).

In this effective clearance are, among others, the effects of clotting and of a recirculation in the shunt are determined, whereby conclusions related to the therapy performance can be made. In contrast, methods based on changes of the conductivity can only estimate the dialyzer clearance.

$${Q}_{Clearance} = ( \frac{{A}_{Dialysate\_Standardized} \cdot {Q}_{Dialysate\_Standard}}{{A}_{Blood}} ) \qquad ( 7 )$$

FIG. 1 shows: graphic context of the clearance of a dialyzer and the blood flow.

1-10. (canceled)

11. An apparatus for extracorporeal treatment of blood comprising:
a dialyzer which is separated by a semipermeable membrane into a first and a second chamber, wherein the first chamber is disposed in a dialysis fluid pathway and the second chamber is connectable to the blood circulation of a patient by means of a blood inflow conduit and a blood outflow conduit;
a feed for fresh dialysis fluid;
a discharge for spent dialysis fluid;
a means for determining a flow Q_{Blood} in the blood inflow conduit or the blood outflow conduit;
a means for determining a flow Q_{Dialysate} in the dialysis fluid pathway;
a measuring device disposed in the discharge for estimation of the absorption A_{Dialysate} of the spent dialysis fluid flowing through the discharge, wherein the measuring device has at least one radiation source and a detector system for detection of the intensity of the electromagnetic radiation;
a memory, wherein the formula
$${A}_{Blood} = ( \frac{{A}_{Dialysate} \cdot {Q}_{Dialysate}}{{Q}_{Blood}} )$$
is stored for the absorption A_{Blood}; and
an arithmetic unit for estimation of the absorption A_{Blood} in the blood inflow conduit or the blood outflow conduit using the absorption A_{Dialysate}, the flow Q_{Dialysate}, and the flow Q_{Blood}.

12. Apparatus according to claim 11, wherein the arithmetic unit is a microprocessor.

13. Apparatus according to claim 11, wherein the radiation source emits a substantially narrow-banded electromagnetic radiation.

14. Apparatus according to claim 11, wherein the radiation source emits a substantially monochromatic electromagnetic radiation.

15. Apparatus according to claim 11, wherein the radiation source is a light emitting diode.

16. Apparatus to claim 11, wherein the means for estimation of a flow Q_{Blood} in the blood inflow conduit or the blood outflow conduit and/or the means for estimation of a flow Q_{Dialysate} in the dialysis fluid pathway are designed for the estimation of flows between 0 ml/min and 1000 ml/min.

17. Apparatus according to claim 11, characterized in that the means for estimation of a flow Q_{Blood} in the blood inflow conduit or the blood outflow conduit and/or the means for estimation of a flow Q_{Dialysate} in the dialysis fluid pathway are designed as a pump with adjustable flow rate or as a direct flow measuring device.

18. Apparatus according to claim 11, wherein the arithmetic unit is provided for estimation of the equilibrated eKt/V-value using the formula
$$eKt / V = - ln ( \frac{{A}_{Blood\_end of therapy}}{{A}_{Blood\_starting point}} ) .$$

19. Apparatus according to claim 11, wherein the arithmetic unit is provided for estimation of the absorption of the medium molecules in the blood which is proportional to the concentration of the medium molecules in the blood using the formula
$${A}_{Blood\_MM} = ( {A}_{Standard + UF} - {A}_{Standard} ) \cdot ( \frac{{Q}_{Blood}}{{Q}_{UF}} ) .$$

20. Apparatus according to claim 11, wherein the arithmetic unit is provided for estimation of the equilibrated eKt/V-value for the medium molecular proportion using the formula
$$eKt / {V}_{convection} = - ln ( \frac{{A}_{Blood\_MM \_end of therapy}}{{A}_{Blood\_MM \_starting point}} ) .$$
